(19) United States
(12) Patent Application Publication
(10) Pub. No.: US 2004/0156800 A1
(43) Pub. Date: Aug. 12, 2004

(54) Cosmetic composition comprising at least one cyclic carbonate

(76) Inventors: Gaelle Brun, Paris (FR); Isabelle Rollat-Corvol, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/702,441

(22) Filed: Nov. 7, 2003

Related U.S. Application Data
(60) Provisional application No. 60/434,654, filed on Dec. 20, 2002.

(30) Foreign Application Priority Data
Nov. 7, 2002 (FR) 02 13938

(51) Int. Cl. A61K 7/04; A61K 7/06; A61K 7/11

(52) U.S. Cl. 424/61; 424/70.11

(57) ABSTRACT

A cosmetic composition comprising, in a cosmetically acceptable medium, at least one cyclic carbonate capable of polymerizing under an external stimulus, in combination with at least one compound chosen from anionic fixative polymers, nonionic fixative polymers, amphoteric fixative polymers, conditioning agents, anionic direct dyes, quaternized cationic direct dyes, oxidation dyes, reducing agents, surfactants, and oxidizing agents, and method for treatment of hair, eyelashes, skin, and/or nails using said composition.

This application claims benefit of U.S. Provisional Application No. 60/434,654, filed Dec. 20, 2002.

Disclosed herein is a cosmetic composition comprising, in a cosmetically acceptable medium, (i) at least one compound chosen from certain fixative polymers, conditioning agents, certain direct dyes, oxidation dyes, reducing agents, surfactants, and oxidizing agents and (ii) at least one cyclic carbonate capable of polymerizing under an external stimulus. Also disclosed herein is the use of these cyclic carbonates for the treatment of hair, skin, nails, and/or skin, for example, to give hair body, volume, or fullness.

As used herein, “treatment of hair, skin, nails, and/or skin” means any cosmetic action that modifies the properties of one of these materials, such as hair. These properties may be, for example, physicochemical or sensory in nature.

As used herein, “composition to give hair body, volume, or fullness” means a composition which may have the effect of increasing the overall volume of the hairstyle, while giving the hair strength and resilience. “Hair styling composition” means a composition for hair intended for maintaining and/or fixing the hairstyle.

As used herein, “cyclic carbonate” means a salt or ether salt of a carboxylic acid having at least one C₃ to C₃₅ ring.

Cosmetic hair products that give hair fullness are already known. These products, based on film-forming polymers, may be valued, for example, by users with fine hair. However, one of their limitations is the fact that the fullness may disappear after the first shampoo. Furthermore, these products may make the hair feel rough, as a result of polymer build-up on the surface of the hair.

Permanent waving can also be done to give hair fullness. Such permanent waves, based on the use of reducing and oxidizing agents, require that the hair be placed under mechanical tension, for example by means of rollers. Even if such treatments effectively increase the hair volume, nevertheless they may have the disadvantage of altering the state of the hair fiber.

Similarly, while coloring products already exist that can give hair body, it would be desirable for them to also provide a better styling effect, such as one that is more durable, or for them to do less damage to the state of the hairs.

It would also likewise be desirable for shampoos to give the hair more volume and better cosmetic properties while retaining considerable cleaning power.

It would also be generally desirable to realize cosmetic compositions that last longer when the hair is shampooed than the currently available compositions.

The documents EP 1 022 014, U.S. Pat. No. 4,746,322, U.S. Pat. No. 4,402,985, and DE 198 51 451 describe the use of some cyclic carbonates in cosmetics, for example serving as solvents. Nevertheless, the cyclic carbonates described in those documents are not capable of polymerizing under an external stimulus.

Some methods have been described for giving hair lasting body and bulk, for example for fine hair. The most commonly described method is polymerization of a molecule inside the fiber. However, the systems described often use toxic precursors, and the working conditions required for polymerization, for example oxidation, may cause appreciable degradation of the fiber.

Thus there was a need in the art to find compositions for treatment of hair, and which, for example, would give fullness or styling to hair, while improving the resistance to shampooing, such as after coloring, without having all the disadvantages listed above.

In order to address this problem, the present inventors propose the cosmetic use for treatment of hair, skin, nails, and/or eyelashes, for example in hair cosmetics, in a cosmetically acceptable medium, of at least one cyclic carbonate capable of polymerizing under at least one external stimulus.

The external stimulus may be, for example, chosen from radical polymerization initiators, anionic polymerization initiators, raising of the temperature, lowering of the temperature, and light. In certain embodiments, an anionic polymerization initiator, such as water, may be used.

According to one embodiment, the external stimulus may be the water present on the hair, originating for example from wet hair or the humidity of the air.

The method disclosed herein may, for example, involve hair styling, hair care, washing hair, coloring hair, make-up of hair, or shaping hair, and for example either to give hair body, volume, or fullness, or to improve resistance to shampooing, such as after coloring.

For instance, the presently disclosed method for treatment of hair, eyelashes, skin and/or nails may comprise applying to the hair, eyelashes, skin and/or nails, a composition comprising, in a cosmetically acceptable medium, a compound of formula (1):

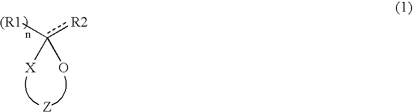

wherein:

X is chosen from O and S;

n is chosen from 0 and 1;

R2 is chosen from O, S, OR3, and SR3;

R1 and R3, which may be identical or different, are chosen from linear and branched C₁ to C₃₀ alkyl radicals, optionally substituted by at least one entity chosen from halogens and from amine, hydroxy, carboxy, C₁ to C₁₀ alkoxy, thiol, C₆ to C₂₀ aryl, and C₁ to C₈ alkoxy groups;

R1 may form a heterocycle together with R2;

Z is chosen from divalent linear or branched C₂ to C₃₀ alkylene radicals, optionally interrupted by at least one heteroatom, and optionally substituted by at least one entity chosen from halogens and from hydroxy, C₆ to C₃₀ aryl, amino, carboxy, C₁ to C₁₀ alkoxy, and thiol groups;

R2 may form a heterocycle together with an atom of Z, the heterocycle being optionally substituted, and optionally comprising at least one heteroatom; and wherein

compounds of formula (I) comprise less than four fused rings.

In one embodiment of formula (I),

If X is O, R2 is O, and n is 0, then Z is not ethylene, isopropylene, propylene, 2-hydroxypropylene, or isopropyl-2,2′-dimethyl propylene.

In formula (I), Z may be chosen from divalent linear or branched C₄ to C₃₀ alkylene radicals, such as C₆ to C₂₈ divalent linear or branched alkylene radicals.

Also disclosed herein is a cosmetic composition comprising, in a cosmetically acceptable medium, (i) at least one compound chosen from anionic fixative polymers, non-ionic fixative polymers, amphoteric fixative polymers, conditioning agents, anionic direct dyes, quaternized cationic direct dyes, oxidation dyes, reducing agents, surfactants, and oxidizing agents with (ii) at least one cyclic carbonate capable of polymerizing under at least one external stimulus.

In certain embodiments of the composition, the at least one cyclic carbonate may be, for example, a compound of formula (1)

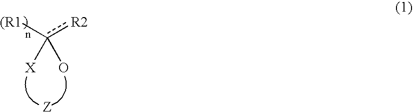

wherein R1, R2, X, Z, and n are chosen as set forth above.

The external stimulus with which the at least one cyclic carbonate is capable of polymerizing may be a polymerization initiator, which may be, for example, chosen from tin octanoate, potassium acetate, and potassium carbonate, optionally combined with at least one of a crown ether, sodium hydride, aluminum trichloride, dibutyltin oxide, aluminum isopropoxide, and diethylzinc.

Compound of Formula (1)

In the compound of formula (1), the oxygen heterocycle comprising Z may be, for example, a heterocycle comprising at least six members.

Z may be, for example, a C₃-C₁₅ radical, such as a C₃-C₁₀ radical, and for example a C₃-C₆ radical.

Compounds of formula (1) may, for example, be chosen from those that satisfy the criteria below, where R and R′, which may be identical or different, are chosen from C₁ to C₄ alkyl radicals:

(i) X is O, n is 0, R2 is S, and Z is CH₂—CH(R);

(ii) X is O, n is 0, R2 is S, and Z is CH₂—CH(R)—CH₂;

(iii) X is S, n is 0, R2 is S, and Z is CH₂—CH(R);

(iv) X is O, n is 1, Z is CH₂—CH₂, and

R1 together with R2 is

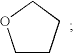

(v) X is O, n is 0, R2 is O, and Z is CH₂—C(R, R′)—CH₂;

(vi) X is O, n is 0, R2 is O, and Z is CH₂—CH₂—CH₂—CH₂;

(vii) X is O, n is 1, Z is CH₂CH₂CH₂, and R1 together with R2 is

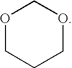

Compounds of the formula (1) may, for example, be those described in the documents below:

Nemoto, N., Xu., X., Sanda, F., and Endo, T.; 34 Macromolecules 7642-7647 (2001);

Kakimoto, K., Nemoto, N., Sanda, F., and Endo, T.; Chemistry Letters 156-157 (2002);

Endo et al.; 157 Macromol. Symp. 21-28 (2000);

Takata, T., Endo, T.; 18 Prog. Polym. Sci, 839-870 (1993);

Endo T. and Sanda F.; 159 Macromol. Symp. 1-7 (2000);

Hitomi, M., Xu, X., Sanda, F., and Endo, T.; 200 Macromol. Chem. Phys. 1268-1273 (1999);

Takata, T. et al.; 18 Prog. Polym. Sci. 839-870 (1993);

Sanda et al.; 18 Macromol. Rapid Commun. 461-469 (1997);

Matsuo et al.; 199 Macromol. Chem. Phys. 97-102 (1998); and

Kihara, N. et al.; 31 Synthesis and Properties of Poly(hydroxyurethane)s; J. Polym. Sci. Part A: Polym. Chem. 2765-2773 (1993).

The aforementioned documents also indicate various procedures for preparation of cyclic carbonates according to the invention.

The compound of the formula (1) in the inventive compositions may, for example, be present in an amount ranging from 0.001% to 50%, for example from 0.01% to 30%, or for example from 0.1% to 20% by weight relative to the total weight of the composition.

Without being bound to a theory, it is believed that application of the composition according to certain embodiments disclosed herein may lead to formation, on the surface or between the hairs, of a polymeric or oligomeric material which may be linear, branched, or multiply branched.

The polymeric or oligomeric material formed in this way may reinforce and make hair fuller, and so may have the effect of giving hair texture, reinforcement, bulk, or volume. Assuming that polymerization has occurred inside the fiber, then the polymer formed may be too large to escape from the fiber, which would ensure that these properties last when the hair is shampooed.

The following properties may also be changed by the application of the inventive composition to the hair:

drying speed,

water uptake,

behavior in a humid environment, for example for better resistance of the set, and

behavior when dyed, for example protection of sensitized areas, better uptake and resistance of the color.

When the inventive composition is added to dye compositions, the resistance of the color may also be improved.

It is also possible to modify the feel of the hair, to give a styling effect, and to modify the detangling and drying properties.

The compounds disclosed herein may also permit links between hairs, and an effect of maintaining the hairstyle may be achieved. This effect may be resistant to shampooing.

The polymeric or oligomeric material formed as described above may be depolymerized. In one embodiment, it may be possible to modify or reduce physical properties, such as solidity, insolubility, and surface tension, of the material after it has been formed. It may be possible to reduce the solidity or insolubility of the material by depolymerization, i.e., reverse reaction leading to a decrease in the size of the polymer, or returning it to the monomer state, wherein the monomer then can be in a state where the initial ring is open, using heat. Thus the materials may last through shampooing. This modification may be carried out after application of the process, if it is not desired to keep the effect achieved or considerably later, i.e., after a few hours, a few days or even later if necessary.

Surfactant

The surfactants useful in the compositions of the invention, for example as component (i) as disclosed above, may be chosen from nonionic, cationic, anionic, and amphoteric surfactants.

As examples of anionic surfactants, mention may be made of the salts of the following compounds, such as the alkali metal salts, for example the sodium salts; ammonium salts; the salts of amines; the salts of amino alcohols; and the alkaline-earth metal salts, for example the magnesium salts: alkyl sulfates, alkyl ether sulfates, alkylamide ether sulfates, alkyl aryl polyether sulfates, monoglyceride sulfates, alkyl sulfonates, alkyl phosphates, alkylamide sulfonates, alkyl aryl sulfonates, α-olefin sulfonates, paraffin sulfonates, alkyl sulfosuccinates, alkyl ether sulfosuccinates, alkylamide sulfosuccinates, alkyl sulfoacetates, acyl sarcosinates, and acyl glutamates, where the alkyl or acyl groups of all these compounds comprise from 6 to 24 carbon atoms and the aryl group may, for example, be chosen from phenyl groups and benzyl groups.

C₆-C₂₄ alkyl esters of polyglycoside carboxylic acids may also be used, such as alkyl glucoside citrates, alkyl polyglycoside tartrates, and alkyl polyglycoside sulfosuccinates, alkyl sulfosuccinamates, acyl isethionates, and N-acyl taurates, where the alkyl or acyl group of all of these compounds comprises from 12 to 20 carbon atoms. Among the anionic surfactants that also may be used, mention can be made of acyl lactylates wherein the acyl group comprises from 8 to 20 carbon atoms.

In addition, mention can also be made of alkyl D-galactoside uronic acids, polyalkoxylated (C₆-C₂₄)-alkyl ether carboxylic acids, polyalkoxylated (C₆-C₂₄)-alkyl (C₆-C₂₄)-aryl ether carboxylic acids, polyalkoxylated (C₆-C₂₄)-alkylamide ether carboxylic acids, such as those containing from 2 to 50 ethylene oxide groups, the salts of all of these compounds, and mixtures thereof.

The nonionic surfactants are also known compounds in themselves (in this regard, see for example “Handbook of Surfactants” by M. R. PORTER, Blackie & Son (Glasgow and London), 1991, pp. 116-178). Thus they may be chosen, for example, from alcohols, alpha-diols, (C₁-C₂₀)-alkyl phenols, polyethoxylated fatty acids, polypropoxylated fatty acids, and polyglycerolated fatty acids, wherein the fatty chain has, for example, from 8 to 18 carbon atoms, and where the number of ethylene oxide or propylene oxide groups may range, for example, from 2 to 50, and the number of glycerol groups may range, for example, from 2 to 30. Mention can also be made of copolymers of ethylene oxide and propylene oxide, condensates of ethylene and propylene oxide with fatty alcohols, polyethoxylated fatty amides, for example having from 2 to 30 moles ethylene oxide, polyglycerolated fatty amides containing on the average from 1 to 5 and for example from 1.5 to 4 glycerol groups, ethoxylated sorbitan fatty acid esters having from 2 to 30 moles ethylene oxide, sucrose fatty acid esters, polyethylene glycol fatty acid esters, (C₆-C₂₄)-alkyl polyglycosides, N-(C₆-C₂₄)-alkyl glucamine derivatives, amine oxides such as (C₁₀-C₁₄)-alkyl amine oxides, N-(C₁₀-C₁₄)-acylaminopropylmorpholine oxides, and mixtures thereof.

The amphoteric surfactants may be, for example, derivatives of secondary or tertiary aliphatic amines wherein the aliphatic group is chosen from linear or branched chains comprising from 8 to 22 carbon atoms and comprising at least one anionic water-solubilizing group such as, for example, carboxylate, sulfonate, sulfate, phosphate, and phosphonate groups. Mention can also be made of (C₈-C₂₀)-alkyl betaines, sulfobetaines, (C₈-C₂₀)-alkylamide (C₆-C₈)-alkyl betaines, (C₈-C₂₀)-alkylamide (C₆-C₈)-alkyl sulfobetaines, and mixtures thereof.

Among the amine derivatives, mention can be made of the products marketed under the name MIRANOL®, such as those described in the U.S. Pat. Nos. 2,528,378 and 2,781,354, and listed in the CTFA [Cosmetic, Toiletry, and Fragrance Association] Dictionary, 3^{rd} edition, 1982, under the names Amphocarboxyglycinate and Amphocarboxypropionate, corresponding respectively to formulas (a) and (b):

R₄—CONHCH₂CH₂—N⁺(R₅)(R₆)(CH₂COO⁻) (a)

wherein:

R₄ represents an alkyl group derived from an R₄—COOH acid present in at least one group chosen from hydrolyzed coconut oil, heptyl groups, nonyl groups, and undecyl groups;

R₅ represents a beta-hydroxyethyl group; and

R₆ represents a carboxymethyl group;

and

R₄′—CONHCH₂CH₂—N(B)(C) (b)

wherein:

B represents —CH₂CH₂OX′;

C represents —(CH₂)_{z}—Y′, wherein z is chosen from 1 and 2;

X′ is chosen from —CH₂CH₂—COOH groups and hydrogen atoms;

Y′ is chosen from —COOH groups and —CH₂—CHOH—SO₃H groups;

R₄′ is chosen from alkyl groups of an R₄′—COOH acid present in coconut oil, alkyl groups of an R₄′—COOH acid present in hydrolyzed linseed oil, alkyl groups, such as C₁₇ alkyl groups, the iso form of C₁₇ alkyl groups, and unsaturated C₁₇ groups.

These compounds are listed in the CTFA Dictionary, 5^{th} edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

As an example, there can be mentioned the cocoamphodiacetate sold by RHODIA under the trade name MIRANOL® C2M concentrate.

One embodiment of the composition disclosed herein may comprise at least one cationic surfactant that is known in itself, such as salts of primary, secondary, or tertiary fatty amines, optionally polyalkoxylated, quaternary ammonium salts, and mixtures thereof.

As the quaternary ammonium salts, mention can be made of, for example,

those having the general formula (VI) below:

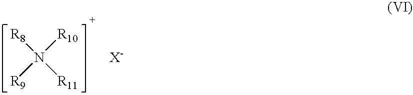

wherein the radicals R₈, R₉, R₁₀, and R₁₁, which can be identical or different, are chosen from linear or branched aliphatic radicals having from 1 to 30 carbon atoms, and aromatic radicals such as aryls and alkyl aryls. The aliphatic radicals may include heteroatoms, such as for example oxygen, nitrogen, sulfur, and halogens. The aliphatic radicals may be, for example, chosen from alkyl, alkoxy, (C₂-C₆)-polyoxyalkylene, alkylamide, (C₁₂-C₂₂)-alkylamide (C₂-C₆)-alkyl, (C₁₂-C₂₂)-alkyl acetate, and hydroxyalkyl radicals, said radicals having from 1 to 30 carbon atoms; X is an anion chosen from halides, phosphates, acetates, lactates, (C₂-C₆)-alkyl sulfates, alkyl sulfonates, and alkyl aryl sulfonates;

quaternary ammonium salts of imidazoline, such as, for example, those of formula (VII) below:

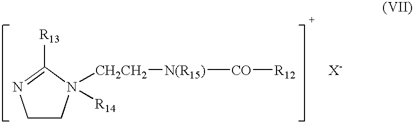

wherein R₁₂ is chosen from alkenyl radicals having from 8 to 30 carbon atoms and alkyl radicals having from 8 to 30 carbon atoms, and for example, derived from fatty acids of tallow; R₁₃ is chosen from hydrogen atoms, C₁-C₄ alkyl radicals, alkenyl radicals having from 8 to 30 carbon atoms, and alkyl radicals having from 8 to 30 carbon atoms; R₁₄ represents a C₁-C₄ alkyl radical; R₁₅ is chosen from hydrogen atoms and C₁-C₄ alkyl radicals; and X⁻ is an anion chosen from halides, phosphates, acetates, lactates, alkyl sulfates, alkyl sulfonates, and alkyl aryl sulfonates. For example, R₁₂ and R₁₃ may be a mixture of alkenyl or alkyl radicals having from 12 to 21 carbon atoms, for example, derivatives of fatty acids of tallow; R₁₄ may be a methyl radical; and R₁₅ may be a hydrogen atom. Such a product is sold, for example, by REWO under the name REWOQUAT® W;

quaternary diammonium salts of formula (VIII):

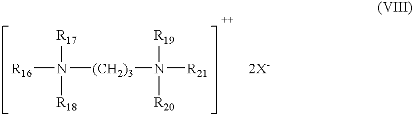

wherein R₁₆ is an aliphatic radical having about from 16 to 30 carbon atoms; R₁₇, R₁₈, R₁₉, R₂₀, and R₂₁, which may be identical or different, are chosen from hydrogen atoms and an alkyl radicals having from 1 to 4 carbon atoms; and X is an anion chosen from halides, acetates, phosphates, nitrates, and methyl sulfates. Such quaternary diammonium salts may, for example, include tallow propane diammonium dichloride;

quaternary ammonium salts comprising at least one ester functional group, such as those of formula (IX) below:

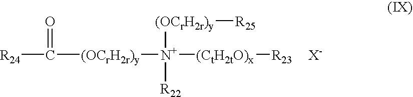

wherein:

R₂₂ is chosen from C₁-C₆ alkyl radicals, C₁-C₆ hydroxyalkyl radicals, and C₁-C₆ dihydroxyalkyl radicals;

R₂₃ is chosen from:

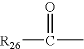

linear or branched, saturated or unsaturated C₁-C₂₂ hydrocarbon radicals R₂₇, and

hydrogen atoms,

R₂₅ is chosen from:

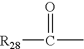

linear or branched, saturated or unsaturated C₁-C₆ hydrocarbon radicals R₂₉, and

hydrogen atoms,

R₂₄, R₂₆, and R₂₈, which may be identical or different, are chosen from linear or branched, saturated or unsaturated C₇-C₂₁ hydrocarbon radicals;

r, s, and t, which may be identical or different, are integers ranging from 2 to 6;

y is an integer ranging from 1 to 10;

x and z, which may be identical or different, are integers ranging from 0 to 10;

X⁻ is chosen from organic simple anions, organic complex anions, inorganic simple anions, and inorganic complex anions;

provided that the sum of x+y+z has a value ranging from 1 to 15 if x equals 0, then R₂₃ is R₂₇, and if z equals 0 then R₂₅ is R₂₉.

The alkyl radicals R₂₂ may be chosen from linear or branched radicals, for example linear radicals.

R₂₂ may, for example, be chosen from methyl radicals, ethyl radicals, hydroxyethyl radicals, and dihydroxypropyl radicals, for example methyl radicals and ethyl radicals.

The sum of x+y+z may, for example, have a value ranging from 1 to 10.

When R₂₃ is a hydrocarbon radical R₂₇, it may be long and have from 12 to 22 carbon atoms, or it may be short and have from 1 to 3 carbon atoms.

When R₂₅ is a hydrocarbon radical R₂₉, it may have, for example, 1 to 3 carbon atoms.

R₂₄, R₂₆, and R₂₈, which may be identical or different, may, for example, by chosen from linear or branched, saturated or unsaturated C₁₁-C₂₁ hydrocarbon radicals, or for example from linear or branched, saturated or unsaturated C₁₁-C₂₁alkyl and alkenyl radicals.

x and z, which may be identical or different, may be, for example, chosen from 0 and 1.

And y may be, for example, equal to 1.

r, s, and t, which may be identical or different, may be, for example, chosen from 2 and 3, and may be, for example, 2.

The anion X⁻ may, for example, be chosen from halides, such as chloride, bromide, and iodide, and alkyl sulfates, such as methyl sulfate. Methanesulfonate, phosphate, nitrate, tosylate, anions derived from organic acids such as acetate and lactate, and any other anion compatible with ammonium comprising an ester functional group may be used.

The anion X⁻ may, for example, be chosen from chloride sulfate and methyl sulfate.

In certain embodiments of the composition, ammonium salts of formula (IX) may be used wherein:

R₂₂ is chosen from methyl radicals and ethyl radicals;

x and y are equal to 1;

z is chosen from 0 and 1;

r, s, and t are equal to 2;

R₂₃ is chosen from:

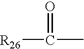

methyl radicals, ethyl radicals, and C₁₄-C₂₂ hydrocarbon radicals; and

hydrogen atoms;

R₂₅ is chosen from:

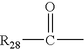

hydrogen atoms;

R₂₄, R₂₆, and R₂₈, which may be identical or different, are chosen from linear or branched, saturated or unsaturated C₁₃-C₁₇ hydrocarbon radicals, for example linear or branched, saturated or unsaturated C₁₃-C₁₇ alkyl and alkenyl radicals.

The hydrocarbon radicals, may, for example, be linear.

For example, mention can be made of compounds of formula (IX), for example salts, such as chloride sulfate salts and methyl sulfate salts, diacyloxyethyl dimethyl ammonium, diacyloxyethyl hydroxyethyl methyl ammonium, monoacyloxy ethyl dihydroxyethyl methyl ammonium, triacyloxyethyl methyl ammonium, monoacyloxyethyl hydroxyethyl dimethyl ammonium, and mixtures thereof. The acyl radicals for example may have 14 to 18 carbon atoms and for example may come from a vegetable oil such as palm or sunflower oil. When the compound comprises more than one acyl radical, the acyl radicals may be identical or different.

These products may be obtained, for example, by direct esterification of compounds chosen from optionally alkoxylated triethanolamine, triisopropanolamine, alkyl diethanolamine, and alkyl diisopropanolamine with plant and animal fatty acids and mixtures of plant and animal fatty acids, and by transesterification of their methyl esters. This esterification may be followed by quaternization using an alkylation agent such as an alkyl halide, for example methyl halide and ethyl halide, a dialkyl sulfate, for example methyl and ethyl, methyl methanesulfonate, methyl para-toluenesulfonate, glycol chlorohydrin, and glycerol chlorohydrin.

Such compounds are, for example, sold by HENKEL under the names DEHYQUART®, STEPANQUAT® sold by STEPAN, NOXAMIUM® sold by CECA, and REWOQUAT® WE 18 sold by REWO-WITCO.

The composition disclosed herein may comprise a mixture of monoester, diester, and triester quaternary ammonium salts, with diester salts comprising the greatest part by weight.

For example, a mixture of ammonium salts that may be used is the mixture comprising from 15% to 30% by weight acyloxyethyl dihydroxyethyl methyl ammonium methyl sulfate, from 45% to 60% by weight diacyloxyethyl hydroxyethyl methyl ammonium methyl sulfate, and from 15% to 30% by weight triacyloxyethyl methyl ammonium methyl sulfate, where the acyl radicals have 14 to 18 carbon atoms and are derived from palm oil, optionally partially hydrogenated.

The ammonium salts comprising at least one ester functional group that are described in U.S. Pat. Nos. 4,874,554 and 4,137,180 may also be used.

Among the quaternary ammonium salts of formula (VI), certain embodiments may use tetraalkylammonium chlorides such as, for example, dialkyl dimethyl ammonium chlorides and alkyl trimethyl ammonium chlorides wherein the alkyl radical has 12 to 22 carbon atoms, such as, for example, behenyl trimethyl ammonium, distearyl dimethyl ammonium, cetyl trimethyl ammonium, and benzyl dimethyl stearyl ammonium chlorides. In other embodiments, palmityl amidopropyl trimethyl ammonium chloride and stearamidopropyl dimethyl (myristyl acetate) ammonium chloride sold by VAN DYK under the name CERAPHYL® 70 may be used.

The cationic surfactants according to certain embodiments of the composition may be chosen from quaternary ammonium salts, such as, for example, from among behenyl trimethyl ammonium chloride and palmitylamidopropyl trimethyl ammonium chloride.

Anionic or Nonionic or Amphoteric Fixative Polymer

As used herein, “fixative polymer” means a polymer capable of maintaining and/or fixing the hairstyle in a desired shape.

The anionic fixative polymers which may be used herein, may, for example, be chosen from polymers including groups derived from, for example, carboxylic acid, sulfonic acid, or phosphoric acid, and having a number-average molecular weight ranging from 500 to 5,000,000.

The carboxylic groups are contributed by unsaturated monocarboxylic or dicarboxylic acid monomers such as those corresponding to the formula:

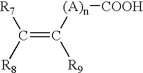

wherein

n is an integer ranging from 0 to 10;

A is a methylene group, optionally linked to a carbon atom of the unsaturated group or to the adjacent methylene group when n is greater than 1, via the intermediary of a heteroatom, such as oxygen or sulfur;

R₇ is chosen from hydrogen atoms, phenyl groups, and benzyl groups;

R₈ is chosen from hydrogen atoms, lower alkyl groups, and carboxyl groups; and

R₉ is chosen from hydrogen atoms, lower alkyl groups, —CH₂—COOH, phenyl groups, and benzyl groups.

In the aforementioned formula, a lower alkyl group may, for example, be a group having 1 to 4 carbon atoms, for example methyl and ethyl groups.

Anionic fixative polymers comprising carboxylic groups according certain embodiments of the invention may be, for example, chosen from:

A) Homopolymers or copolymers of acrylic acid or methacrylic acid or their salts, such as, for example, the products sold by ALLIED COLLOID under the names VERSICOL® E or K and by BASF under the name ULTRAHOLD®, the copolymers of acrylic acid and acrylamide sold by HERCULES as the sodium salt under the names RETEN® 421, 423, and 425, and the sodium salts of polyhydroxycarboxylic acids.

B) Copolymers of acrylic or methacrylic acid with a monoethylene monomer such as ethylene, styrene, vinyl esters, esters of acrylic or methacrylic acid, optionally grafted on a polyalkylene glycol such as polyethylene glycol, and optionally crosslinked. Such polymers are described, for example, in the French patent FR 1 222 944 and the German patent application DE 2 330 956, where the chain of copolymers of this type comprises at least one acrylamide unit, optionally N-alkylated and optionally N-hydroxyalkylated, such as described for example in the Luxembourg Patent Application Nos. 75370 and 75371 and sold under the name QUADRAMER® by AMERICAN CYANAMID. Mention can also be made of copolymers of acrylic acid and C₁-C₄ alkyl methacrylate and terpolymers of vinylpyrrolidone, acrylic acid, and C₁-C₂₀ alkyl methacrylate, for example, lauryl methacrylate, such as those sold by ISP under the name ACRYLIDONE® LM, and methacrylic acid/ethyl acrylate/tert-butyl acrylate terpolymers such as the product sold under the name LUVIMER® 100 P by BASF.

C) Copolymers derived from crotonic acid, such as those comprising in their chain, units chosen from vinyl acetate units and propionate units, and optionally other monomers such as allyl esters, methallyl esters, vinyl ethers of a saturated carboxylic acid, vinyl esters of a saturated carboxylic acid, where the copolymers may be linear or branched, with a large hydrocarbon chain, such as those having at least 5 carbon atoms, wherein these polymers can optionally be grafted and crosslinked, and may comprise also another monomer chosen from vinyl, allyl or methallyl esters of α- or β-cyclic carboxylic acids. Such polymers are described, inter alia, in the French patents FR 1 222 944, FR 1 580 545, FR 2 265 782, FR 2 265 781, FR 1 564 110, and FR 2 439 798. Commercial products included in this class are, for example, the resins 28-29-30, 26-13-14, and 28-13-10 sold by NATIONAL STARCH.

D) Copolymers chosen from derivatives of mono-unsaturated C₄-C₈ carboxylic acids and mono-unsaturated C₄-C₈ carboxylic anhydrides chosen from:

copolymers comprising (i) at least one of maleic acid, fumaric acid, itaconic acid, maleic anhydride, fumaric anhydride, and itaconicanhydride, and (ii) at least one monomer chosen from vinyl esters, vinyl ethers, vinyl halides, phenyl vinyl derivatives, and acrylic acid and its esters, where the anhydride functional groups of these copolymers are optionally monoesterified or monoamidified. Such polymers are described for example in U.S. Pat. Nos. 2,047,398, 2,723,248, and 2,102,113 and British patent GB 839 805. Commercial products are for example those sold under the names GANTREZ® AN and ES by ISP.

copolymers comprising (i) at least one of maleic units, citraconic units, and itaconic units, and (ii) at least one monomer chosen from allyl esters and methallyl esters, optionally comprising at least one of acrylamide groups, methacrylamide groups, α-olefin groups, acrylic esters, methacrylic esters, acrylic acids, methacrylic acids, and vinylpyrrolidone in their chain, where the anhydride functional groups of these copolymers are optionally monoesterified or monoamidified.

These polymers are described, for example, in the French patents FR 2 350 384 and FR 2 357 241.

E) Polyacrylamides comprising sulfonic groups.

The polymers comprising sulfonic groups may be, for example, polymers comprising at least one of vinylsulfonic, styrenesulfonic, naphthalenesulfonic, and acrylamide alkyl sulfonic units.

These polymers may be chosen, for example, from:

salts of polyvinylsulfonic acid having a molecular weight ranging from 1,000 to 100,000, as well as copolymers with an unsaturated comonomer, such as acrylic acids or methacrylic acids and their esters, as well as acrylamide and its derivatives, vinyl ethers, and vinylpyrrolidone.

salts of polystyrene sulfonic acid such as the sodium salts sold, for example, under the names FLEXAN® 130 and FLEXAN® 500 by NATIONAL STARCH. These compounds are described, for example, in the French patent FR 2 198 719.

salts of polyacrylamide sulfonic acids, such as those mentioned in U.S. Pat. No. 4,128,631, and for example polyacrylamide ethyl propanesulfonic acid sold under the name COSMEDIA POLYMER® HSP 1180 by HENKEL.

Amphoteric fixative polymers that may be used may be chosen from polymers comprising units B and C statistically distributed within the polymer chain, where B is a unit derived from a monomer comprising at least one basic nitrogen atom and C is a unit derived from a monomer comprising at least one of carboxylic groups and sulfonic groups. The amphoteric fixative polymers may also comprise at least one of carboxybetaine zwitterionic units and sulfobetaine zwitterionic units.

Mention can also be made of polymers with a cationic main chain comprising at least one of primary, secondary, tertiary, and quaternary amine groups, among which at least one bears, via the intermediary of a hydrocarbon radical, a group chosen from carboxylic acids and sulfonic acids. The amphoteric fixative polymers may also comprise an anionic chain derived from α,β-unsaturated carboxylic acids where one of the carboxyl groups has been reacted with a polyamine comprising at least one primary amine group.

Amphoteric fixative polymers corresponding to the definition given above may be chosen, for example, from the following polymers:

(1) polymers resulting from copolymerization of at least one monomer derived from a vinyl compound bearing a carboxylic group, such as for example acrylic acid, methacrylic acid, maleic acid, and alpha-chloroacrylic acid, and at least one basic monomer derived from a substituted vinyl compound comprising at least one basic atom, such as for example dialkylamino alkyl methacrylate, dialkylamino alkyl acrylate, dialkylamino alkyl methacrylamide, and dialkylamino alkyl acrylamide. Such compounds have been described, for example, in U.S. Patent No. 3,836,537.

(2) polymers comprising units deriving from:

(a) at least one monomer chosen from acrylamides and methacrylamides substituted on the nitrogen atom by an alkyl group,

(b) at least one acid comonomer comprising at least one reactive carboxylic group; and

(c) at least one basic comonomer, such as esters substituted by primary, secondary, tertiary, and quaternary amines of acrylic acid and methacrylic acid, and the product of quaternization of dimethylaminoethyl methacrylate with a sulfate chosen from dimethyl sulfates and diethyl sulfates.

The N-substituted acrylamides or methacrylamides may be, for example, compounds where the alkyl groups have 2 to 12 carbon atoms, for example N-ethyl acrylamide, N-tert-butyl acrylamide, N-tert-octyl acrylamide, N-octyl acrylamide, N-decyl acrylamide, and N-dodecyl acrylamide as well as the corresponding methacrylamides.

The acid comonomers may be, for example, chosen from acrylic acid, methacrylic acid, crotonic acid, itaconic acid, maleic acid, fumaric acid, alkyl monoesters, wherein the alkyl group has 1 to 4 carbon atoms, of maleic acids and anhydrides, and alkyl monoesters, wherein the alkyl group has 1 to 4 carbon atoms, of fumaric acids and anhydrides.

The basic comonomers according to certain embodiments may be, for example, chosen from aminoethyl methacrylates, butylaminoethyl methacrylates, N,N′-dimethylaminoethyl methacrylates, and N-tert-butylaminoethyl methacrylates.

The copolymers that may be used are, for example, those for which the CTFA name (4th edition, 1991) is octyl acrylamide/acrylates/butylaminoethyl methacrylate copolymer, such as the products sold under the name AMPHOMER® or LOVOCRYL® 47 by NATIONAL STARCH.

(3) crosslinked and partially or totally alkylated polyaminoamides derived from polyaminoamides of the general formula:

—(CO—R₁₀—CO-Z-)-

wherein R₁₀ represents a divalent group chosen from derivatives of saturated dicarboxylic acid, derivatives of aliphatic monocarboxylic, derivatives of dicarboxylic acid with a carbon-carbon double bond, esters of a lower alkanol, having 1 to 6 carbon atoms, of these acids, and derivatives of the addition of any one of said acids with at least one of bis-primary amines and bis-secondary amines, and Z denotes a group derived from at least one polyalkylene polyamine, such as the group chosen from bis-primary, mono-secondary, and bis-secondary polyalkylene polyamines, and for example may represent:

a) present in an amount ranging from 60 to 100 mole %, the groups

—NH—[(CH₂)X—NH]ₚ—

where x is 2 and p is chosen from 2 and 3, or where x is 3 and p is 2,

wherein this group is derived from at least one of diethylenetriamines, triethylenetetraamines, and dipropylenetriamines;

b) present in an amount ranging from 0 to 40 mole %, the groups

—NH—[(CH₂)ₓ—NH]ₚ—

where x is 2 and p is 1 and wherein this group is chosen from derivatives of ethylenediamine and derivatives of piperazine:

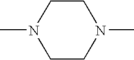

c) present in an amount ranging from 0 to 20 mole %, the groups

—NH—(CH₂)₆—NH—

derived from hexamethylenediamine,

these polyaminoamides being crosslinked by an addition reaction with a bifunctional crosslinking agent, chosen from epihalohydrins, diepoxides, dianhydrides, and bis-unsaturated derivatives, using 0.025 to 0.35 moles of crosslinking agent per amine group of the polyaminoamide, and being alkylated by treatment with a compound chosen from acrylic acid, chloroacetic acid, alkane sultone, and salts thereof.

The saturated carboxylic acids may, for example, be chosen from acids having 6 to 10 carbon atoms, such as adipic acid, 2,2,4-trimethyladipic acid, 2,4,4-trimethyladipic acid, terephthalic acid, and acids with a carbon-carbon double bond such as, for example, acrylic acid, methacrylic acid, and itaconic acid.

The alkane sultones used in the alkylation may, for example, be chosen from propane sultone and butane sultone, and the salts of the alkylation agents may be, for example, chosen from sodium salts and potassium salts.

(4) polymers comprising zwitterionic units of the formula:

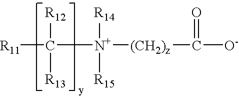

wherein R₁₁ denotes a polymerizable unsaturated group such as a group chosen from acrylate, methacrylate, acrylamide, and methacrylamide; y and z each represent an integer ranging from 1 to 3; R₁₂ and R₁₃ are chosen from hydrogen atoms, methyl groups, ethyl groups, and propyl groups; R₁₄ and R₁₅ are chosen from hydrogen atoms and alkyl groups such that the sum of the carbon atoms in R₁₄ and R₁₅ is not greater than 10.

The polymers comprising such units may also comprise units derived from nonzwitterionic monomers, such as monomers chosen from dimethylaminoethyl acrylate, diethylaminoethyl acrylate, dimethylaminoethyl methacrylayte, diethylaminoethyl methacrylate, alkyl acrylates, alkyl methacrylates, acrylamides, methacrylamides, and vinyl acetate.

As an example, mention may be made of methyl methacrylate/methyl dimethyl carboxymethyl ammonium ethyl methacrylate copolymers, such as the product sold under the name DIAFORMER® Z301 by SANDOZ.

(5) polymers derived from chitosan, comprising monomer units corresponding to the following formulas:

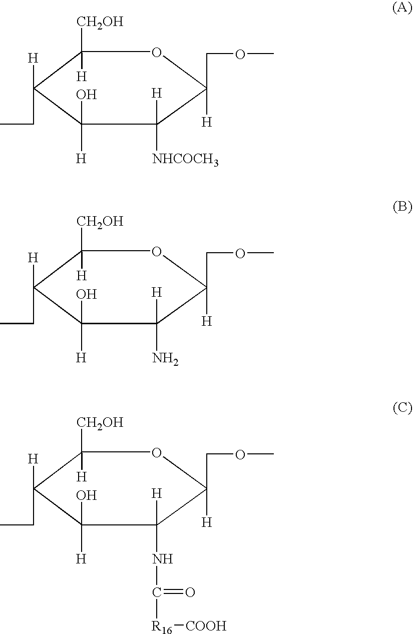

where unit (A) is present in an amount ranging from 0% to 30%, unit (B) is present in an amount ranging from 5% to 50%, and unit (C) is present in an amount ranging from 30% to 90%, where it is understood that in unit (C), R₁₆ represents a group of formula:

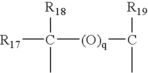

wherein if q is 0, R₁₇, R₁₈ and R₁₉, which may be identical or different, are chosen from hydrogen atoms, and from methyl, hydroxyl, acetoxy residues, amino, monoalkylamine, and dialkylamine residues, optionally interrupted by at least one nitrogen atom and optionally substituted by at least one group chosen from amine, hydroxyl, carboxyl, alkylthio, and sulfonic groups, and alkylthio residues, where the alkyl group bears an amino residue,

wherein at least one of the groups R₁₇, R₁₈, and R₁₉ is a hydrogen atom; and

wherein if q is 1, R₁₇, R₁₈, and R₁₉ each represent a hydrogen atom, as well as the salts formed by these compounds with bases or acids.

(6) Polymers obtained by N-carboxylation of chitosan, such as the polymers chosen from N-carboxymethyl chitosan and N-carboxybutyl chitosan sold under the name EVALSAN® by JAN DEKKER.

(7) Polymers described in the French patent FR 1 400 366 and corresponding to the formula

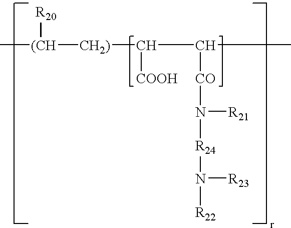

wherein R₂₀ is chosen from hydrogen atoms, CH₃O, CH₃CH₂O, and phenyl groups; R₂₁ is chosen from hydrogen atoms and lower alkyl groups, such as methyl and ethyl; R₂₂ is chosen from hydrogen atoms and C₁-C₆ lower alkyl groups such as methyl and ethyl; R₂₃ is chosen from C₁-C₆ lower alkyl groups such as methyl and ethyl groups, and groups corresponding to the formula: —R₂₄—N(R₂₂)₂, where R₂₄ is chosen from —CH₂—CH₂—, —CH₂—CH₂—CH₂—, and —CH₂—CH(CH₃)— groups, and where R₂₂ has the meanings indicated above.

(8) Amphoteric polymers of the type -D-X-D-X, chosen from:

(a) the polymers obtained by treatment, with chloroacetic acid or sodium chloroacetate, of compounds comprising at least one unit of formula:

-D-X-D-X-D-

where D denotes a

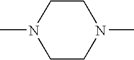

group and X is chosen from the symbols E and E′; E and E′, which may be identical or different, denote a divalent group which is chosen from straight-chain or branched alkylene groups, having up to 7 carbon atoms in the main chain, optionally substituted by hydroxyl groups, and which may additionally include oxygen, nitrogen, sulfur atoms, 1 to 3 aromatic rings, and 1 to 3 heterocyclic rings; where the oxygen, nitrogen, and sulfur atoms are present in the form of at least one group chosen from ether, thioether, sulfoxide, sulfone, sulfonium, alkylamine, alkenylamine, hydroxyl, benzylamine, amine oxide, quaternary ammonium, amide, imide, alcohol, ester, and/ urethane groups.

(b) Polymers of formula:

-D-X-D-X—

where D denotes a

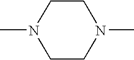

group and X denotes the symbol E and E′; and E′ appears at least once; where E has the meaning indicated above and E′ is a divalent group which is chosen from straight-chain or branched alkylene groups, having up to 7 carbon atoms in the main chain, optionally substituted by at least one hydroxyl group and comprising at least one nitrogen atom, where the nitrogen atom is substituted by an alkyl chain, optionally interrupted by an oxygen atom and necessarily comprises at least one of carboxyl functional groups and hydroxyl functional groups, and betainized by reaction with at least one of chloroacetic acid and sodium chloroacetate.

(9) (C₁-C₅)-alkyl vinyl ether/maleic anhydride copolymers modified partially by half-amidification with an N,N-dialkylamino alkyl amine such as N,N-dimethylaminopropylamine or by half-esterification with an N,N-dialkanol amine. These copolymers may also comprise other vinyl comonomers, such as vinylcaprolactam.

The nonionic fixative polymers may be chosen from, for example, from:

vinylpyrrolidone,

copolymers of vinylpyrrolidone and vinyl acetate,

polyalkyloxazolines, such as the polyethyloxazolines sold by DOW CHEMICAL under the names PEOX® 50 000, PEOX® 200 000, and PEOX® 500 000,

homopolymers of vinyl acetate, such as the product sold under the name APPRETAN® EM by HOECHST and the product sold under the name RHODOPAS® A 012 by RHONE POULENC,

copolymers of vinyl acetate and acrylic ester, such as the product sold under the name RHODOPAS®AD 310 by RHONE POULENC,

copolymers of vinyl acetate and ethylene, such as the product sold under the name APPRETAN® TV by HOECHST,

copolymers of vinyl acetate and maleic ester, for example, dibutyl maleate, such as the product sold under the name APPRETAN® MB EXTRA by HOECHST,

copolymers of polyethylene and maleic anhydride,

poly(alkyl acrylate) and poly(alkyl methacrylate), such as the product sold under the name MICROPEARL® RQ 750 by MATSUMOTO and the product sold under the name LUHYDRAN® A 848 S by BASF,

copolymers of acrylic esters, such as, for example, copolymers of alkyl acrylates and alkyl methacrylates, such as the products sold by ROHM & HAAS under the names PRIMAL® AC-261 K and EUDRAGIT® NE 30 D, by BASF under the names ACRONAL® 601, LUHYDRAN® LR 8833 and 8845, by HOECHST under the names APPRETAN® N9212 and N9213;

copolymers of acrylonitrile and a nonionic monomer selected, for example, from among butadiene and alkyl (meth)acrylates; mention can also be made of the products sold under the names NIPOL® LX 531 B by NIPPON ZEON and those sold under the name CJ 0601 B by ROHM & HAAS,

polyurethanes, such as the products sold under the names ACRYSOL® RM 1020 and ACRYSOL® RM 2020 by ROHM & HAAS, the products URAFLEX® XP 401 and URAFLEX® XP 402 UZ from DSM RESINS,

polyamides, such as the product ESTAPOR® LO 11 offered by RHONE POULENC,

optionally chemically modified nonionic guar gums. The unmodified guar gums may be, for example, the products sold under the name VIDOGUM® GH 175 by UNIPECTINE and under the name JAGUAR® C by MEYHALL.

The modified guar gums may be, for example, modified by C₁₋₆ hydroxyalkyl groups, for example by hydroxymethyl, hydroxyethyl, hydroxypropyl, and hydroxybutyl groups. Such nonionic guar gums, optionally modified by hydroxyalkyl groups, are sold, for example, under the trade names JAGUAR® HP8, JAGUAR® HP60, JAGUAR® HP120, JAGUAR® DC 293, and JAGUAR® HP 105 by MEYHALL, and under the name GALACTOSOL® 4H4FD2 by AQUALON.

Fixative polymers that may also be used include film-forming polymers such as grafted silicone comprising a polysiloxane portion and a portion made up of a non-silicone organic chain, one of the two portions comprising the main chain of the polymer and the other being grafted on said main chain.

These polymers are described, for example, in the patent applications EP A 0 412 704, EP A 0 412 707, EP A 0 640 105, WO 95/00578, EP A 0582 152 WO 93/23009, and U.S. Pat. Nos. 4,693,935, 4,728,571, and 4,972,037.

These polymers may be, for example, chosen from anionic polymers and nonionic polymers.

Such polymers are, for example, copolymers that can be obtained by radical polymerization starting from a mixture of monomers formed from:

a) 50% to 90% by weight tert-butyl acrylate,

b) 0% to 40% by weight acrylic acid,

c) 5% to 40% by weight of a silicone macromer of formula

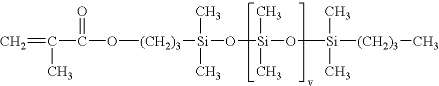

where v is a number ranging from 5 to 700, and where the weight percents are calculated relative to the total weights of the monomers.

Other examples of grafted silicone polymers are for example polydimethylsiloxanes (PDMS) on which are grafted, via the intermediary of a thiopropylene type linkage, mixed polymer units of the poly(meth)acrylic acid type and the alkyl poly(meth)acrylate type and polydimethylsiloxanes (PDMS) on which are grafted, via the intermediary of a thiopropylene type linkage, polymer units of the isobutyl poly(meth)acrylate type.

Fixative polymers that may be used in the styling compositions of certain embodiments may also comprise functionalized or nonfunctionalized, siliconized or unsiliconized polyurethane.

The polyurethanes useful herein may be, for example, those described in the documents EP A 0 751 162, EP A 0 637 600, FR 2 743 297, EP 0 648 485, EP A 0 656 021, WO 94/03510, and EP A 0 619 111.

Conditioning Agent

As used herein, “conditioning agent” means a compound that may be capable of giving hair cosmetic properties, such as softness to the touch, smoothness, or detangling properties.

The conditioning agent may, for example, be chosen from organosiloxanes, linear or branched C₈ to C₃₀ hydrocarbons, linear or branched C₈ to C₃₀ fatty alcohols, esters of C₈ to C₃₀ fatty acids and C₁ to C₃₀ alcohols, esters of C₁ to C₇ acids or diacids and C₈ to C₃₀ fatty alcohols, ceramides, ceramide analogs, and cationic polymers.

Among the organosiloxides used according according to certain embodiments, mention can be made without limitation thereto of the following:

I. Volatile Silicones

These may have a boiling point ranging from 60° C. to 260° C. Among this type of silicone, mention can be made of the following:

(i) cyclic silicones having 3 to 7 silicon atoms, and for example, 4 to 5 silicon atoms. This may include, for example, the octamethyl cyclotetrasiloxane sold under the name “VOLATILE SILICONE 7207®” by UNION CARBIDE and “SILBIONE 70045 V2®” sold by RHÔNE POULENC, the decamethyl cyclopentasiloxane sold under the name “VOLATILE SILICONE 7158®” by UNION CARBIDE, “SILBIONE 70045 V5®” from RHÔNE POULENC, and mixtures thereof.

Mention can also be made of cyclopolymers of the dimethylsiloxane/methylalkyl siloxane type, such as the “SILICONE VOLATILE FZ 3109®” sold by UNION CARBIDE, which is a dimethylsiloxane/methyloctylsiloxane cyclocopolymer;

(ii) linear volatile silicones having 2 to 9 silicon atoms, and having a viscosity less than or equal to 5·10⁻⁶ m²/s at 25° C. For example, this includes the hexamethyldisiloxane sold under the name “SILBIONE 70041 V0,65®” by RHÔNE POULENC. This type of product is described in the article by TODD & BYERS, “Volatile silicone fluids for cosmetics,” Cosmetics and Toiletries, Vol. 91, January 1976, pages 27-32.

II. Nonvolatile Silicones

These may be mainly made up of poly((C₁-C₃₅)-alkyl siloxanes), poly(aryl siloxanes), poly((C₁-C₃₅)-alkyl aryl siloxanes), silicone gums and resins, organomodified polysiloxanes, and mixtures thereof.

Among the polyalkyl siloxanes, mention can be made, for example, of linear polydimethylsiloxanes with a viscosity greater than 5·10⁻⁶ m²/s, and for example, less than 2.6 m²/s:

either with terminal trimethylsilyl groups such as, for example, without limitation thereto, “SILBIONE®” oils of the 70047 series sold by RHÔNE POULENC, the “47 V 500.000®” oil sold by RHÔNE POULENC, and “VISCASIL®” sold by GENERAL ELECTRIC,

or with terminal trihydroxysilyl groups, such as oils of the “48 V®” series sold by RHÔNE POULENC.

Within this class of polyalkylsiloxanes, mention can also be made of polyalkyl siloxanes sold by GOLDSCHMIDT under the names “ABILWAX 9800®)” and “ABILWAX 9801®”, which are poly-(C₁-C₂₀)-alkyl siloxanes.

Among the poly(alkyl aryl siloxanes), mention can be made of linear or branched polydimethylphenylsiloxanes, polydimethyidiphenylsiloxanes, with viscosity ranging from 10⁻⁵ to 5·10⁻² m²/s, such as, for example:

“RHODORSIL®” 763 oil sold by RHÔNE POULENC,

“SILBIONE®” oils of the 70641 series sold by RHÔNE POULENC, “SILBIONE 70641 V30®”, and “SILBIONE 70641 V200®” oils sold by RHÔNE POULENC,

the product “DC 556®” Cosmetic Grad Fluid sold by DOW CORNING,

silicones of the PK series sold by BAYER, such as “PK20®”,

silicones of the PN, PH series sold by BAYER, such as “PN 1000®” and “PH 1000®”,

some oils in the SF series sold by GENERAL ELECTRIC, such as “SF 1250®”, “SF 1265®”, ∓SF 1154®”, and “SF 1023®”.

Silicone gums according to certain embodiments may be polydiorganosiloxanes with a high number-average molecular weight, ranging from 200,000 to 1,000,000, used alone or in a mixture in a solvent chosen from volatile silicones, polydimethylsiloxane oils (PDMS), polyphenylmethylsiloxane oils (PPMS), isoparaffins, methylene chloride, pentane, dodecane, tridecane, tetradecane, and mixtures thereof.

For example, mention can be made of compounds with the following structures:

poly[(dimethylsiloxane)/(methylvinylsiloxane)],

poly[(dimethylsiloxane)/(diphenylsiloxane)],

poly[(dimethylsiloxane)/(phenylmethylsiloxane)], and

poly[(dimethylsiloxane)/(diphenylsiloxane)/(methylvinylsiloxane)].

The following mixtures can be mentioned, without limitation thereto:

1 ) mixtures formed from an end-hydroxylated polydimethylsiloxane (DIMETHICONOL according to CTFA nomenclature) and a cyclic polydimethylsiloxane (CYCLOMETHICONE according to CTFA nomenclature), such as the product “Q2 1401®” sold by DOW CORNING;

2) mixtures formed from a polydimethylsiloxane gum with a cyclic silicone, such as the product “SF 1214 SILICONE FLUID®” sold by GENERAL ELECTRIC, which is a “SE 30®” gum with MW 500 000 (Mn) solubilized in “SF 1202 SILICONE FLUID®” (decamethylcyclopentasiloxane);

3) mixtures of at least two PDMSs of different viscosities, for example at least one PDMS gum and at least one PDMS oil, such as the products “SF 1236®” and “CF 1241®” sold by GENERAL ELECTRIC. The product “SF 1236®” is the mixture of an “SE 30®” gum specified above with a viscosity of 20 m²/s and an “SF 96®” oil with a viscosity of 5·10⁻⁶ m²/s (15% “SE 30®” gum and 85% “SF 96®” oil).

The product “CF 1241®” is the mixture of one “SE 30®” gum (33%) and one PDMS (67%) with viscosity 10⁻³ m²/s.

Organopolysiloxanes that can be used may be, for example, crosslinked siloxane systems comprising the units: R₂SiO_{2/2}, RSiO_{3/2}, and SiO_{4/2}, wherein R is chosen from hydrocarbon groups having 1 to 6 carbon atoms and phenyl groups. Among these products, mention may be made of those wherein R is chosen from lower alkyl radicals and phenyl radicals.

Among these resins, mention can be made of the product sold under the name “DOW CORNING 593®” and those sold under the names “SILICONE FLUID SS 4230” and “SILICONE FLUID SS 4267“by GENERAL ELECTRIC and which are dimethyl/trimethylpolysiloxanes.

Organomodified silicones according to certain embodiments may be silicones such as those specified above, comprising in their general structure at least one organofunctional group directly attached to the siloxane chain or attached via the intermediary of a hydrocarbon radical.

For example, mention can be made of silicones comprising:

a) perfluorinated groups, such as trifluoroalkyls such as, for example, those sold by GENERAL ELECTRIC under the names “FF.150 FLUOROSILICONE FLUID®” and sold by SHIN ETSU under the names “X-22-819®”, “X-22-82®”, “X-22-821®”, and “X-22-822®”;

b) hydroxyacylamino groups such as, for example, those described in the patent application EP A 0 342 834 and, for example, the silicone sold by DOW CORNING under the name “Q2-8413®”;

c) thiol groups such as in the silicones “X 2-8360®” sold by DOW CORNING and “GP 72A®” and “GP 71®” sold by GENESEE;

d) optionally substituted amine groups, such as in “GP 4 SILICONE FLUID®” sold by GENESEE, “GP 7100®” sold by GENESEE, “Q2 8220®” sold by DOW CORNING, “AFL 40®” sold by UNION CARBIDE, and the silicone named “Amodimethicone” in the CTFA Dictionary;

e) carboxylate groups, such as the products described in the patent EP 186 507;

f) hydroxylated groups, such as polyorganosiloxanes with a hydroxyalkyl functional group, described in the French patent application FR 85 16334, corresponding to the formula below:

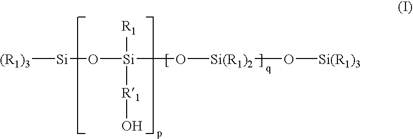

wherein:

the R₁ radicals, which may be identical or different, are chosen from methyl radicals and phenyl radicals, wherein at least 60 mole % of the R₁ radicals are methyl;

the R′₁ radical is a divalent C₂-C₁₈ alkylene hydrocarbon group;

p ranges from 1 to 30; and

q ranges from 1 to 150.

Mention can be made, for example, of the product sold by Dow Corning under the name DC 190;

g) alkoxyl groups such as in the silicone copolymer “F 755®” sold by SWS SILICONES and the products “ABILWAX 2428®”, “ABILWAX 2434®”, and “ABILWAX 2440®” sold by GOLDSCHMIDT;

h) acyloxyalkyl groups, such as for example the polyorganosiloxanes described in French patent FR 88 17433, corresponding to the formula below:

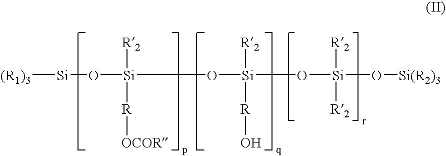

wherein:

R₂ is chosen from methyl, phenyl, OCOR″, and hydroxyl, wherein only one R₂ per silicon atom may be OH;

R′₂ is chosen from methyl and phenyl, wherein at least 60 mole % of all the R₂ and R′₂ radicals are methyl;

R″ is chosen from C₈-C₂₀ alkyls and C₈-C₂₀ alkenyls;

R is chosen from divalent linear or branched C₂-C₁₈ hydrocarbon alkylenes;

r ranges from 1 to 120;

p ranges from 1 to 30; and

q is chosen from 0 and numbers less than 0.5 p, wherein p+q ranges from 1 to 30:

wherein the polyorganosiloxanes of formula (II) may comprise

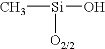

groups in proportions no greater than 15% of the sum p+q+r;

i) quaternary ammonium groups, such as in the products “X2 81 08®” “X2 81 09®”, and “ABIL K 3270®” sold by GOLDSCHMIDT;

j) groups chosen from amphoteric groups and betaine groups, such as in the product sold by GOLDSCHMIDT under the name “ABIL B 9950®;

k) bisulfite groups, such as in the products sold by GOLDSCHMIDT under the names “ABIL S 201®” and “ABIL S 255®”.

The viscosities of silicones may be determined for example by the standard ASTM D445-97 (viscometry).

When the conditioning agent of the composition according to certain embodiments is a hydrocarbon, it is chosen from linear or branched C₈-C₃₀ hydrocarbons. Among the hydrocarbons that are liquid at room temperature and match this definition, mention can be made of, for example, isododecane, isohexadecane and its isomers, such as 2,2,4,4,6,6,8-heptamethylnonane, isoeicosane, isotetracosane, and isomers of said compounds. According to the invention, for example, isododecane and one of its isomers may be used.

When the conditioning agent is a fatty alcohol, it may be chosen from linear or branched, saturated or unsaturated C₈-C₃₀ fatty alcohols, and for example, mention can be made of 2-butyloctanol, octyldodecanol, lauryl alcohol, oleyl alcohol, isocetyl alcohol, isostearyl alcohol, and behenyl alcohol.

When the conditioning agent is a fatty ester, it may be chosen from esters of C₈-C₃₀ fatty acids and C₁-C₃₀ alcohols, and esters of C₁-C₇ acids, diacids and C₈-C₃₀ fatty alcohols. Among these esters, mention can be made of ethyl, isopropyl palmitate, ethyl-2-hexyl palmitate, 2-octyldecyl palmitate, isopropyl myristate, butyl myristate, cetyl myristate, 2-octyldecyl myristate, butyl stearate, hexyl stearate, hexyl laurate, 2-hexyldecyl laurate, isononyl isononanoate, and dioctyl malate.

Ceramides and analogs, such as the glycoceramides that can be used in the compositions disclosed herein, are known in themselves and may be chosen from natural and synthetic molecules that may correspond to the general formula below:

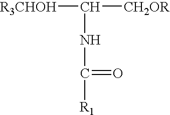

wherein:

R₁ is chosen from linear or branched, saturated or unsaturated radicals derived from C₁₄-C₃₀ fatty acids, wherein this radical is optionally substituted by a hydroxyl group at the a position and optionally substituted a hydroxyl group at the ω position, esterified by a fatty acid chosen from saturated C₁₆-C₃₀ fatty acid and unsaturated C₁₆-C₃₀ fatty acids;

R₂ is chosen from hydrogen atoms, (glycosyl)ₙ, (galactosyl)ₘ, and sulfogalactosyl radicals, wherein n is an integer ranging from 1 to 4 and m is an integer ranging from 1 to 8;

R₃ is chosen from a C₁₅-C₂₆ hydrocarbon radical, optionally saturated at the α position, wherein this radical is optionally substituted by at least one C₁-C₁₄ alkyl radical;

it being understood that in the case of natural ceramides and glycoceramides, R₃ may also be a C₁₅-C₂₆ α-hydroxyalkyl radical, wherein the hydroxyl group is optionally esterified by a C₁₆-C₃₀ α-hydroxy acid.

The ceramides that may be used may be, for example, compounds for which R₁ is chosen from saturated and unsaturated alkyls derived from C₁₆-C₂₂ fatty acids; R₂ denotes a hydrogen atom; and R₃ denotes a linear saturated C₁₅ radical.

Such compounds may be, for example:

N-linoleoyl dihydrosphingosine,

N-oleoyl dihydrosphingosine,

N-palmitoyl dihydrosphingosine,

N-stearoyl dihydrosphingosine,

N-behenoyl dihydrosphingosine, and

mixtures of these compounds.

One may use, for example, ceramides wherein R₁ is chosen from saturated and unsaturated alkyl radicals derived from fatty acids; R₂ is chosen from galactosyl radicals and sulfogalactosyl radicals; and R₃ denotes a

—CH═CH—(CH₂)₁₂—CH₃ group.

Cationic polymers that may be used, include, for example, those chosen from polymers including primary, secondary, tertiary, and/or quaternary amine groups that are part of the polymer chain or are directly linked to it, and that have a number-average molecular weight ranging from 500 to 5,000,000, and, for example, ranging from 1,000 to 3,000,000.

Among these polymers, mention can be made of, for example, the following cationic polymers:

(1) homopolymers and copolymers derived from at least one of acrylic and methacrylic esters and amides, and comprising units corresponding to at least one of the formulas below:

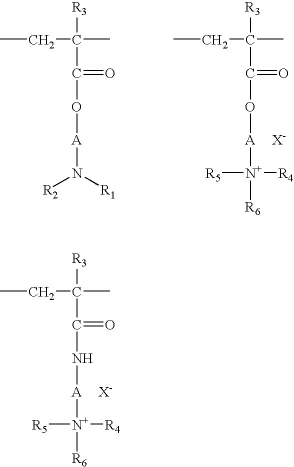

wherein:

R₁ and R₂, which may be identical or different, are chosen from hydrogen atoms and C₁₋₆ alkyl groups;

R₃ is chosen from hydrogen atoms and CH₃ groups;

A is chosen from linear or branched alkyl groups having 1 to 6 carbon atoms, and hydroxyalkyl groups having 1 to 4 carbon atoms;

R₄, R₅, R₆, which may be identical or different, are chosen from alkyl groups having from 1 to 18 carbon atoms and benzyl groups;

X is chosen from methosulfate anions and halides such as chloride and bromine.

Copolymers in family (1) may additionally comprise at least one unit derived from comonomers chosen from acrylamides, methacrylamides, diacetone acrylamides, acrylamides substituted on the nitrogen by lower alkyl groups, methacrylamides substituted on the nitrogen by lower alkyl groups, (methyl)acrylic acids, esters of (methyl)acrylic acids, vinyllactams such as vinylpyrrolidone and vinylcaprolactam, and vinyl esters.

Thus among copolymers of family (1), mention can be made of, for example, the following:

copolymers of acrylamide and dimethylaminoethyl methacrylate, quaternized to the dimethyl sulfate or with a methyl halide, such as that sold under the name HERCOFLOC® by HERCULES,

copolymers of acrylamide and methacryloyloxyethyl trimethyl ammonium chloride described, for example, in the patent application EP A 080976 and sold under the name BINA QUAT® P100 by CIBA GEIGY,

the copolymer of acrylamide and methacryloyloxyethyl trimethyl ammonium methosulfate, sold under the name RETEN® by HERCULES,

copolymers vinylpyrrolidone/dialkylamino alkyl acrylate and vinylpyrrolidone/dialkylamino alkyl methacrylate, optionally quaternized, such as the products sold under the name GAFQUAT® by ISP, such as, for example, GAFQUAT® 734 and GAFQUAT® 755, and the products sold under the name “COPOLYMER® 845, 958, and 937”. These polymers are described in detail in the French patents FR 2 077 143 and FR 2 393 573,

dimethylaminoethyl methacrylate/ vinylcaprolactam/vinylpyrrolidone terpolymers, such as the product sold under the name GAFFIX® VC 713 by ISP, and

vinylpyrrolidone/quaternized dimethylaminopropyl methacrylamide copolymers, such as for example the product sold under the name GAFQUAT® HS 100 by ISP.

(2) the quaternized polysaccharides described for example in U.S. Pat. Nos. 3,589,578 and 4,031,307, such as guar gums comprising cationic trialkyl ammonium groups. Such products are, for example, sold under the names JAGUAR C13 S, JAGUAR C 15, and JAGUAR C 17 by MEYHALL.

(3) quaternary copolymers of vinylpyrrolidone and vinylimidazole, such as the products sold by BASF under the name LUVIQUAT TFC.

(4) chitosans and their salts, such as chitosan acetate, lactate, glutamate, gluconate, and pyrrolidone carboxylate.

Among these compounds, mention can be made of chitosans having a deacetylation rate of 90.5% by weight, sold under the name KYTAN BRUT STANDARD by ABER TECHNOLOGIES, and the chitosan pyrrolidone carboxylate sold under the name KYTAMER® PC by AMERCHOL.

(5) derivatives of cationic cellulose, such as copolymers of cellulose and cellulose derivatives grafted with a water-soluble monomer comprising quaternary ammonium, and described for example in U.S. Pat. No. 4,131,576, such as hydroxyalkyl celluloses, wherein said hydroxyalkyl celluloses can be, for example, hydroxymethylcelluloses, hydroxyethylcelluloses, and hydroxypropylcelluloses grafted for example with a compound chosen from methacryloyloxyethyl trimethylammonium, methacrylamidopropyl trimethylammonium, and dimethyl diallyl ammonium salts.

These polymers are sold for example under the names CELQUAT® L200 and CELQUAT® H100 by NATIONAL STARCH.

(6) Homopolymers and copolymers of dialkyl diallyl ammonium halides, sold for example under the names MERQUAT, MERQUAT 550, and MERQUAT S by Nalco.

(7) Polycondensates based on quaternized diamine units, such as polyquaternium 2, sold under the name MIRAPOLA 15 by Rhodia, and hexadimethrine chloride, sold under the name MEXOMERE PO by Chimex.

Cationic and Anionic Direct Dyes

As used herein, “anionic direct dye” means direct dyes having at least one group chosen from —SO₃X and —CO₂X groups in their structure, wherein X is chosen from hydrogen atoms, alkali metals, alkaline earth metals, and organic amines.

“Quaternized cationic direct dye” means, as used herein, direct dyes having at least one quaternized nitrogen atom.

Cationic and anionic direct dyes that may be used include those, for example, described in the COLOUR INDEX INTERNATIONAL, 3rd edition, under the names BASIC dyes and ACID dyes, and those in the patent applications WO 95/15144, WO 95/01772, and EP 714 954.

Among direct azo dyes, mention can be made of, for example, the following dyes, described in the COLOUR INDEX INTERNATIONAL, 3^{rd} edition:

Acid Yellow 9,

Acid Black 1,

Basic Red 22,

Basic Red 76,

Basic Yellow 57,

Basic Brown 16,

Acid Yellow 36,

Acid Orange 7,

Acid Red 33,

Acid Red 35,

Basic Brown 17,

Acid Yellow 23, and

Acid Orange 24.

Among synthetic quinone direct dyes, mention can be made of, for example, the following compounds:

Acid Violet 43,

Acid Blue 62,

Basic Blue 22,

Basic Blue 99, and

the compound:

-1-N-methylmorpholinium propylamino-4-hydroxyanthraquinone.

Among synthetic azine direct dyes, mention can be made of, for example, the following compounds:

Basic Blue 17 and

Basic Red 2.

Among synthetic triarylmethane direct dyes that may be used, mention can be made of, for example, the following compounds:

Basic Green 1,

Acid Blue 9,

Basic Violet 3,

Basic Violet 14,

Basic Blue 7,

Acid Violet 49,

Basic Blue 26, and

Acid Blue 7.

Oxidation Dye

The oxidation dyes that may be used according to certain embodiments may, for example, be chosen from oxidation bases and couplers.

The compositions disclosed herein, for example, may comprise at least one oxidation base.

Oxidation bases that may be used may, for example, be chosen from those traditionally known in oxidation dyeing, and among them, mention can be made of, for example, ortho- and para-phenylenediamines, double bases, ortho- and para-aminophenols, the heterocyclic bases below, and their acid addition salts.

Mention can be made of, for example:

(I) para-phenylenediamines of formula (I) below and their acid addition salts:

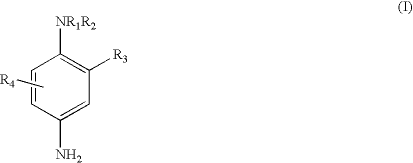

wherein:

R₁ is chosen from hydrogen atoms, C₁-C₄ alkyl, C₁-C₄ monohydroxyalkyl, C₂-C₄ polyhydroxyalkyl, (C₁-C₄)-alkoxy (C₁-C₄)-alkyl, and C₁-C₄ alkyl radicals, substituted by at least one of nitrogen-containing phenyl and nitrogen-containing 4′-aminophenyl;

R₂ is chosen from hydrogen atoms, C₁-C₄ alkyl, C₁-C₄ monohydroxyalkyl, C₂-C₄ polyhydroxyalkyl, (C₁-C₄)-alkoxy (C_{-C}₄)-alkyl, and C₁-C₄ alkyl radicals, substituted by a nitrogen-containing group;

R₁ and R₂ may also form, together with the nitrogen atom to which they are attached, a 5-membered or 6-membered nitrogen heterocycle optionally substituted by at least one of alkyl, hydroxy, and ureido groups;

R₃ is chosen from hydrogen atoms, halogen atoms such as chlorine atoms, and from radicals chosen from C₁-C₄ alkyl, sulfo, carboxy, C₁-C₄ monohydroxyalkyl, C₁-C₄ hydroxyalkoxy, C₁-C₄ acetylaminoalkoxy, C₁-C₄ mesylaminoalkoxy, and C₁-C₄ carbamoylaminoalkoxy radicals,

R₄ is chosen from hydrogen atoms, halogen atoms, and C₁-C₄ alkyl radicals.

Among the nitrogen-containing groups of formula (I) above, mention can be made of, for example, amino radicals, mono-(C₁-C₄)-alkylamino radicals, di-(C₁-C₄)-alkylamino radicals, tri-(C₁-C₄)-alkylamino radicals, monohydroxy-(C₁-C₄)-alkylamino radicals, imidazolinium, and ammonium.

Among the para-phenylenediamines of formula (I) above, mention can be made of, for example, para-phenylenediamine, para-toluylenediamine, 2-chloro para-phenylenediamine, 2,3-dimethyl para-phenylenediamine, 2,6-dimethyl para-phenylenediamine, 2,6-diethyl para-phenylenediamine, 2,5-dimethyl para-phenylenediamine, N,N-dimethyl para-phenylenediamine, N,N-diethyl para-phenylenediamine, N,N-dipropyl para-phenylenediamine, 4-amino N,N-diethyl 3-methylaniline, N,N-bis(β-hydroxyethyl) para-phenylenediamine, 4-N,N-bis(β-hydroxyethyl)amino 2-methylaniline, 4-N,N-bis(β-hydroxyethyl)amino 2-chloroaniline, 2-β-hydroxyethyl para-phenylenediamine, 2-fluoro para-phenylenediamine, 2-isopropyl para-phenylenediamine, N-(β-hydroxypropyl) para-phenylenediamine, 2-hydroxymethyl para-phenylenediamine, N,N-dimethyl 3-methyl para-phenylenediamine, N,N-ethyl(β-hydroxyethyl) para-phenylenediamine, N-(β,γ-dihydroxypropyl) para-phenylenediamine, N-(4′-aminophenyl) para-phenylenediamine, N-phenyl para-phenylenediamine, 2-β-hydroxyethyloxy para-phenylenediamine, 2-β-acetylaminoethyloxy para-phenylenediamine, N-(β-methoxyethyl) para-phenylenediamine, 2-methyl-1-N-β-hydroxyethyl para-phenylenediamine, and their acid addition salts.

Among the para-phenylenediamines of formula (I) above, mentoin may be made, for example, of para-phenylenediamine, para-toluylenediamine, 2-isopropyl para-phenylenediamine, 2-β-hydroxyethyl para-phenylenediamine, 2-β-hydroxyethyloxy para-phenylenediamine, 2,6-dimethyl para-phenylenediamine, 2,6-diethyl para-phenylenediamine, 2,3-dimethyl para-phenylenediamine, N,N-bis(β-hydroxyethyl) para-phenylenediamine, 2-chloro para-phenylenediamine, and their acid addition salts.

(II) The term “double bases” as used herein mean compounds comprising at least two aromatic rings bearing at least one of amino groups and hydroxyl groups.

Among the double bases that may be used as oxidation bases in the dyeing compositions, mention may be made of, for example, compounds corresponding to formula (II) below and their acid addition salts:

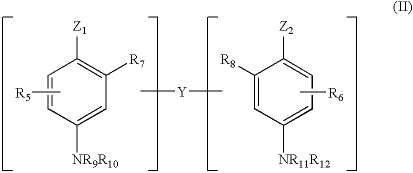

wherein:

Z₁ and Z₂, which may be identical or different, are chosen from hydroxyl radicals and —NH₂ radicals, optionally substituted by at least one of C₁-C₄ alkyl radicals an a linker arm Y;

the linker arm Y is chosen from linear and branched alkylene chains having 1 to 14 carbon atoms and optionally interrupted and optionally terminated by at least one nitrogen-containing group and optionally by at least one heteroatom, such as oxygen, sulfur, and nitrogen atoms, and optionally substituted by at least one of hydroxyl radicals and C₁-C₆ radicals;

R₅ and R₆ are chosen from hydrogen atoms, halogen atoms, C₁-C₄ alkyl, C₁-C₄ monohydroxyalkyl, C₂-C₄ polyhydroxyalkyl, and C₁-C₄ aminoalkyl radicals, and a linker arm Y;

R₇, R₈, R₉, R₁₀, R₁₁, and R₁₂, which may be identical or different, are chosen from hydrogen atoms, a linker arm Y, and C₁-C₄ alkyl radicals;

it being understood that the compounds of formula (II) do not have more than one linker arm Y per molecule.

Among the nitrogen-containing groups of formula (II) above, mention can be made of, for example, amino radicals, mono-(C₁-C₄)-alkylamino radicals, di-(C₁-C₄)-alkylamino radicals, tri-(C₁-C₄)-alkylamino radicals, monohydroxy-(C₁-C₄)-alkylamino radicals, imidazolinium, and ammonium.

Among the double bases of formulas (II) above, mention can be made of, for example, N,N′-bis(β-hydroxyethyl) N,N′-bis(4′-aminophenyl) 1,3-diaminopropanol, N,N′-bis(β-hydroxyethyl) N,N′-bis(4′-aminophenyl) ethylenediamine, N,N′-bis(4-aminophenyl) tetramethylenediamine, N,N′-bis(β-hydroxyethyl) N,N′-bis(4-aminophenyl) tetramethylenediamine, N,N′-bis(4-methylaminophenyl) tetramethylenediamine, N,N′-bis(ethyl) N,N′-bis(4′-amino-3′-methylphenyl) ethylenediamine, 1,8-bis(2,5-diaminophenoxy) 3,5-dioxaoctane, and their acid addition salts.

Among these double bases of formula (II), mention may be made of, for example, N,N′-bis(β-hydroxyethyl) N,N′-bis(4′-aminophenyl) 1,3-diaminopropanol, 1,8-bis(2,5-diaminophenoxy) 3,5-dioxaoctane and their acid addition salts.

(III) para-aminophenols corresponding to formula (III) below, and their acid addition salts:

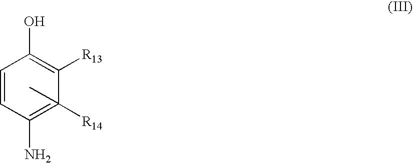

wherein:

R₁₃ is chosen from hydrogen atoms, halogen atoms such as fluorine, and radicals chosen from C₁-C₄ alkyl, C₁-C₄ monohydroxyalkyl, (C₁-C₄)-alkoxy (C₁-C₄)-alkyl, C₁-C₄ aminoalkyl, and (C₁-C₄)-hydroxyalkyl (C₁-C₄)-aminoalkyl radicals;

R₁₄ is chosen from hydrogen atoms, halogen atoms such as fluorine, and radicals chosen from C₁-C₄ alkyl, C₁-C₄ monohydroxyalkyl, C₂-C₄ polyhydroxyalkyl, C₁-C₄ aminoalkyl, C₁-C₄ cyanoalkyl, and (C₁-C₄)-alkoxy (C₁-C₄)-alkyl radicals;

Among the para-aminophenols of formula (III) below, mention can be made of, for example, para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethyl-aminomethyl)phenol, and their acid addition salts.

(IV) the ortho-aminophenols that may be used as oxidation bases, may, for example, be chosen from 2-aminophenol, 2-amino-1-hydroxy-5-methylbenzene, 2-amino-1-hydroxy-6-methylbenzene, 5-acetamido-2-aminophenol, and their acid addition salts.

(V) among the heterocyclic bases that may be used as oxidation basesmention can be made of, for example, pyridine derivatives, pyrimidine derivatives, pyrazole derivatives, and their acid addition salts.

Among the pyridine derivatives, mention can be made of, for example, the compounds described in the British patents GB 1 026 978 and GB 1 153 196, as well as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and their acid addition salts.

Among the pyrimidine derivatives, mention can be made of, for example, the compounds described, for example, in the German patent DE 2 359 399 or the Japanese patents JP 88-169 571 and JP 91-10659 or the patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and pyrazolopyrimidine derivatives such as those mentioned in the patent application FR A 2 750 048, and among those mention can be made of, for example, pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol; 2-(7-aminopyrazolo[1,5-a]pyrimidin-3-ylamino)ethanol; 2-[(3-aminopyrazolo[1,5-a]pyrimidin-7-yl)-(2-hydroxyethyl)amino]ethanol; 2-[(7-aminopyrazolo[1,5-a]pyrimidin-3-yl)-(2-hydroxyethyl)amino]ethanol; 5,6-dimethylpyrazolo[1,5-a]pyrimidine-3,7-diamine; 2,6-dimethylpyrazolo[1,5-a]pyrimidine-3,7-diamine; 2, 5, N7, N7-tetramethylpyrazolo[1,5-a]pyrimidine-3,7-diamine; 3-amino-5-methyl-7-imidazolylpropylaminopyrazolo[1,5-a]pyrimidine; their tautomeric forms, when a tautomeric equilibrium exists; and the acid addition salts.

Among the pyrazole derivatives, mention can be made of, for example, the compounds described in the German patents DE 3 843 892 and DE 4 133 957, and the patent applications WO 94/08969, WO 94/08970, FR A 2 733 749, and DE 195 43 988, such as 4,5-diamino-1-methylpyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino 1-methyl-3-phenyl-pyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-(β-hydroxyethyl)pyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and their acid addition salts.

The couplers that may be used in the dyeing process disclosed herein may be, for example, those traditionally used in oxidation dyeing compositions, that is: meta-aminophenols, meta-phenylenediamines, meta-diphenols, naphthols and heterocyclic couplers such as, for example, indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines and their acid addition salts.

These couplers may be chosen, for example, from 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2-amino 4-(β-hydroxyethylamino)-1-methoxybenzene, 1,3-diaminobenzene, 1,3-bis(2,4-diaminophenoxypropane, sesamol, 1-amino-2-methoxy-4,5-methylened ioxybenzene, α-naphthol, 6-hydroxyindole, 4-hydroxyindole, 4-hydroxy-N-methylindole, 6-hydroxyindoline, 2,6-dihydroxy-4-methylpyridine, 1H-3-methylpyrazol-5-one, 1-phenyl-3-methylpyrazol-5-one, 2-amino-3-hydroxypyridine, 3,6-dimethyl-pyrazolo[3,2-c]-1,2,4-triazole, 2,6-dimethylpyrazolo[1,5-b]-1,2,4-triazole, and their acid addition salts.

Reducing Agent

The reducing agent may, for example, be chosen from thioglycolic acid and its salts, thiolactic acid and its salts, cysteine, cysteamine, glycerol thioglycolate, sulfites, bisulfites, sulfinates, ascorbic acid, erythorbic acid, and glucose.

Oxidizing Agent

The oxidizing agent may, for example, be chosen from hydrogen peroxide, urea peroxide, alkali metal bromates, and persalts such as perborates, persulfates, and periodates.

The at Least One Compound

The at least one compound chosen from anionic fixative polymers, nonionic fixative polymers, amphoteric fixative polymers, conditioning agents, anionic direct dyes, cationic direct dyes, oxidation dyes, reducing agents, surfactants, and oxidizing agents may be present in the composition in an amount ranging from, for example, 0.001% to 50%, or for example from 0.01% to 20%, or as a further example, from 0.1% to 10%.

The composition may additionally comprise, for example, at least one compound chosen from pH control agents, polymerization initiators such as metal salts, polymerization catalysts such as enzymes, solvents such as ethanol, antibacterial agents, pearling agents, propellant gases, particles, nanoparticles, and pigments.

In the composition disclosed herein, the oxidation dye may for example be an oxidation base, or for example it may comprise at least one coupler.

As used herein, the direct dye may be chosen from azo dyes, quinone dyes, azine dyes, and triarylmethane dyes.

The process disclosed herein may, for example, comprise application of the composition to keratinous matter, in particular to hair. This may include, for example, compositions for coloring, bleaching, styling, shampooing, and permanent waving; compositions for use before or after permanent waving, and compositions for use before or after coloring.

According to a particular embodiment, the composition may be realized at the time of use. It may be applied and then rinsed off, applied and not rinsed off, and applied with or without a standing time and with or without heat.

Application of the composition may be followed or preceded by application of other compositions. Rinsing or shampooing can be optionally done between the different applications.

The compositions disclosed herein may be packaged in any conventional device. Aerosol devices may be used that comprise propellants which are insoluble or partially soluble in the liquid phase, such as dimethyl ether, C₃₋₅ alkanes, 1,1-difluoroethane, mixtures of dimethyl ether and C₃₋₅ alkanes, and mixtures of at least two of 1,1-difluoroethane and dimethyl ether, and C₃₋₅ alkanes.

As the propellant, mention may be made, for example, use of C₃₋₅ alkanes, for example propane, n-butane, and isobutane.

The composition may also be provided in the form of lotions, gels, creams, mousses, and sticks.

The composition disclosed herein may be applied for example on hair, but also to skin, eyelashes, and nails.

Particular embodiments may also relate to cosmetic use of at least one cyclic carbonate of formula (1)

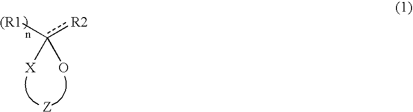

wherein:

X, n, R2, R1, R3, and Z are defined as above, and wherein

the compounds of formula (I) comprise less than four fused rings, for example to give hair volume or fullness or to improve how cosmetic compositions last when the hair is shampooed.

Of course, the person skilled in the art will make sure to select any compound or compounds to add to the composition disclosed herein in such a way that the advantageous properties inherent to the composition selected will not be altered, or not substantially altered, by the contemplated addition.

The formulation example illustrates a particular embodiment but is not limiting thereof.

### EXAMPLE

A composition comprising a compound of formula (I) was applied to a 2.7 g swatch of natural hair, using heat as an activator.

An aqueous solution comprising 10% 5,5′-dimethyidioxan-2-one was applied for 30 minutes,

The swatch was air dried for one hour with no rinsing,

The swatch was dried for 30 minutes at 70° C., and

The swatch was rinsed.

What is claimed is:

1. A method for treatment of at least one of hair, eyelashes, skin and nails, comprising applying to said hair, eyelashes, skin and/or nails a composition comprising, in a cosmetically acceptable medium, at least one cyclic carbonate capable of polymerizing under an external stimulus.

2. The method according to claim 1, wherein the external stimulus is chosen from radical polymerization initiators, anionic polymerization initiators, increased temperature, decreased temperature, and light.

3. The method according to claim 2, wherein the external stimulus is an anionic polymerization initiator.

4. The method according to claim 3, wherein the anionic polymerization initiator is water.

5. The method according to claim 1, wherein said method is a method of treatment for hair.

6. The method according to claim 5, wherein the treatment is chosen from at least one of hair styling, hair care, washing hair, coloring hair, making-up of hair, and shaping hair.

7. The method according to claim 5, wherein the method of treatment provides hair with at least one of body, volume, and fullness.

8. The method according to claim 5, wherein the method of treatment provides hair with improved resistance to shampooing.

9. The method according to claim 8, wherein the method of treatment provides hair with improved resistance to shampooing after the hair is colored.

10. The method according to claim 1, wherein the at least one cyclic carbonate is a compound of formula (1) 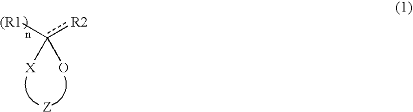
wherein:
X is chosen from O and S;
n is chosen from 0 and 1;
R2 is chosen from O, S, OR3, and SR3;
R1 and R3, which may be identical or different, are chosen from linear or branched C₁ to C₃₀ alkyl radicals, optionally substituted by at least one entity chosen from halogens and from amine, hydroxy, carboxy, C₁ to C₁₀ alkoxy, thiol, C₆ to C₂₀ aryl, and C₁ to C₈ alkoxy groups;
R1 may form a heterocycle together with R2;
Z is chosen from divalent linear or branched C₂ to C₃₀ alkylene radicals, optionally interrupted by at least one heteroatom, and optionally substituted by at least one radical chosen from hydroxys, C₆ to C₃₀ aryls, aminos, halogens, carboxys, C₁ to C₁₀ alkoxys, and thiols; and
R2 optionally forms a heterocycle together with an atom of Z, the heterocycle being optionally substituted, and optionally comprising as least one heteroatom, wherein
the compounds of formula (I) comprise less than four fused rings.

11. The method according to claim 10, wherein Z is chosen from divalent linear or branched C₆ to C₂₈ alkylene radicals.

12. The method according to claim 10, wherein the oxygen heterocycle comprising Z is a heterocycle comprising at least six members.

13. The method according to claim 10, wherein the compound of formula (1) is chosen from compounds which satisfy the criteria below and wherein R and R′ are C₁ to C₄ alkyl radicals:
(i) X is O, n is 0, R2 is S, and Z is CH₂—CH(R);
(ii) X is O, n is 0, R2 is S, and Z is CH₂—CH(R)—CH₂;
(iii) X is S, n is 0, R2 is S, and Z is CH₂—CH(R);
(iv) X is O, n is 1, Z is CH₂—CH₂, and
R1 together with R2 is 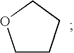
(v) X is O, n is 0, R2 is O, and Z is chosen from CH₂—C(R)—CH₂ and CH₂—C(R′)—CH₂;
(vi) X is O, n is 0, R2 is O, and Z is CH₂—CH₂—CH₂—CH₂;
(vii) X is O, n is 1, Z is CH₂CH₂CH₂, and R1 together with R2 is

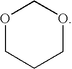

14. The method according to claim 1, wherein the composition further comprises at least one compound chosen from anionic fixative polymers, nonionic fixative polymers, amphoteric fixative polymers, conditioning agents, anionic direct dyes, quaternized cationic direct dyes, oxidation dyes, reducing agents, surfactants, and oxidizing agents.

15. A cosmetic composition comprising, in a cosmetically acceptable medium, (i) at least one compound chosen from anionic fixative polymers, nonionic fixative polymers, amphoteric fixative polymers, conditioning agents, anionic direct dyes, cationic quaternized direct dyes, oxidation dyes, reducing agents, surfactants, and oxidizing agents, combined with (ii) at least one cyclic carbonate capable of polymerizing under an external stimulus.

16. The composition according to claim 15, wherein the at least one cyclic carbonate is a compound of formula (1): 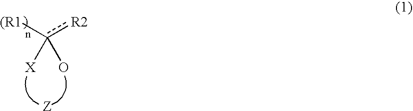
wherein:
X is chosen from O and S;
n is chosen from 0 and 1;
R2 is chosen from O, S, OR3, and SR3;
R1 and R3, which may be identical or different, are chosen from linear or branched C₁ to C₃₀ alkyl radicals, optionally substituted by at least one entity chosen from halogens and from amine, hydroxy, carboxy, C₁ to C₁₀ alkoxy, thiol, C₆ to C₂₀ aryl, and C₁ to C₈ alkoxy groups;
R1 may form a heterocycle together with R2;
Z is chosen from divalent linear or branched C₂ to C₃₀ alkylene radicals, optionally interrupted by at least one heteroatom, and optionally substituted by at least one entity chosen from halogens and from hydroxy, C₆ to C₃₀ aryl, amino, carboxy, C₁ to C₁₀ alkoxy, and thiol groups; and
R2 may form a heterocycle together with an atom of Z, the heterocycle being optionally substituted, and optionally comprising at least one heteroatom; wherein
the compounds of formula (I) comprise less than four fused rings.

17. The composition according to claim 16, wherein Z is chosen from divalent linear or branched C₆ to C₂₈ alkylene radicals.

18. The composition according to claim 16, wherein the at least one cyclic carbonate compound of formula is present in the composition in an amount ranging from 0.001% to 50% by weight, relative to the total weight of the composition.

19. The composition according to claim 18, wherein the at least one cyclic carbonate compound of formula (1) is present in an amount ranging from 0.01% to 30% by weight, relative to the total weight of the composition.

20. The composition according to claim 19, wherein at least one cyclic carbonate compound of formula (1) is present in an amount ranging from 0.1% to 20% by weight, relative to the total weight of the composition.

21. The composition according to claim 15, wherein the at least one compound chosen from anionic fixative polymers, nonionic fixative polymers, amphoteric fixative polymers, conditioning agents, anionic direct dyes, cationic direct dyes, oxidation dyes, reducing agents, surfactants, and oxidizing agents is present in the composition in an amount ranging from 0.001% to 50% by weight, relative to the total weight of the composition.

22. The composition according to claim 21, wherein the at least one compound is present in an amount ranging from 0.01% to 20% by weight, relative to the total weight of the composition.

23. The composition according to claim 22, wherein the at least one compound is present in an amount ranging from 0.1% to 10% by weight, relative to the total weight of the composition.

24. The composition according to claim 15, wherein the reducing agent is chosen from thioglycolic acid and its salts, thiolactic acid and its salts, cysteine, cysteamine, glycerol thioglycolate, sulfites, bisulfites, sulfinates, ascorbic acid, erythorbic acid, and glucose.

25. The composition according to claim 15, wherein the oxidizing agent is chosen from hydrogen peroxide, urea peroxide, alkali metal bromates, and persalts.

26. The composition according to claim 25, wherein said persalts are chosen from perborates, persulfates, and periodates.

27. The composition according to claim 15, wherein the surfactant is chosen from anionic surfactants, cationic surfactants, nonionic surfactants, and amphoteric surfactants.

28. The composition according to claim 15, wherein the oxidation dye is an oxidation base.

29. The composition according to claim 28, further comprising at least one coupler.

30. The composition according to claim 15, wherein the direct dye is chosen from azo dyes, quinone dyes, azine dyes, and triarylmethane dyes.

31. The composition according to claim 15, further comprising at least one additional component chosen from pH control agents, polymerization initiators, polymerization catalysts, solvents, antibacterial agents, pearling agents, propellant gases, particles, nanoparticles, and pigments.

32. The composition according to claim 31, wherein the polymerization initiator is a metal salt.

33. The composition according to claim 31, wherein the polymerization catalysts is an enzyme.

34. The composition according to claim 31, wherein the solvent is ethanol.
